(19) United States
(12) Patent Application Publication
(10) Pub. No.: US 2006/0034794 A1
(43) Pub. Date: Feb. 16, 2006

(54) Odor masking substance and method of making

(76) Inventors: Mark E. Fickle, Buena Vista, CO (US); Ray L. Hauser, Boulder, CO (US)

Correspondence Address: THE INVENTORS NETWORK, INC., 332 ACADEMY STREET, CARNEGIE, PA, 15106, US

(21) Appl. No.: 10/918,842

(22) Filed: Aug. 16, 2004

(51) Int. Cl. A61L 9/01; A61L 9/00

(52) U.S. Cl. 424/76.1

(57) ABSTRACT

An odor masking substance for masking and eliminating various types of noisome smells and odors, such as those that are produced when dressing field game, is created from a number of types of formulations that include an petrolatum based gel, a glycerine/water gel, a water-in-oil emulsion cream, an oil-in-water emulsion cream and a waxy stick similar to deodorant and perfume sticks. The various formulations can be contained within a flexible plastic squeeze tube, and then topically applied about the nose to mask and eliminate the noisome odor.

### FIELD OF THE INVENTION

The present invention pertains to odor masking items, and more particularly pertains to a topically applied substance for masking noisome odors.

### BACKGROUND OF THE INVENTION

Many industries and activities involve the encounter by the individual with odors and smells that are malodorous and noisome. Such industries can include work, employment or involvement with or at sewage treatment facilities, waste disposal landfills, plumbing work, garbage collection, meat processing plants, chemical plants, and petroleum refineries. In addition, one recreational activity where noisome odors are encountered is in hunting, and especially in the process of field dressing game wherein the viscera must be removed by the hunter before the animal can be removed for tagging, weighing, and further processing. In order to mask or conceal the foul odors that are exuded during field dressing of the animal, hunters have previously applied to their nose a petroleum-based product, like Vicks Vapor Rub. Moreover, some hunters have even used such products on the noses of their packhorses to mask or conceal odors emanating from injured or dead animals that could spook the packhorses and cause the horses to bolt and run. Because the concealment, diminution, or elimination of unpleasant, noisome smells and odors is a very desirable quality, devices, substances, and compositions have been conceived to mask or eliminate such odors occurring in a variety of activities and circumstances.

The prior art discloses a number of devices and substances for reducing, masking and eliminating noisome odors.

For example, the Cielo patent (U.S. Pat. No. 5,562,407) discloses an hand-held odor dissipating and removing device that includes a fan enclosed within a container, with the container closed off by barriers, one of which includes a filter, and the device operable by a push button switch to draw air into the housing for reducing the foul odors and then dissipating the odor-reduced air back to the environment.

The Johnson patent (U.S. Pat. No. 6,440,415 B1) discloses the use of activated carbon, in liquid, powder or spray form, for masking human odors during hunting, with such items as soap, laundry detergent, a boot or body powder, and a stick deodorant-antiperspirant containing the composition including the activated carbon.

The Semoff et al. patents (U.S. Pat. No. 5,679,334, U.S. Pat. No. 6,071,506 and U.S. Pat. No. 6,294,162 B1) disclose a gel air freshener that includes a strong, transparent gel air freshener composed from an aqueous gel, a fragrance, a surfactant, and a co-solvent, and that can evaporate over a predetermined time period in order to exude the desired fragrance to the ambient environment.

### SUMMARY OF THE INVENTION

The present invention comprehends various embodiments of compositions or formulations for odorants that can mask or eliminate unpleasant and noisome smells and odors encountered in industries and activities, and with an especial emphasis on masking or reducing odors that accompany the field dressing of game animals. The different types of formulations or compositions can include an oil-base (petrolatum); a glycerine/water gel; a water-in-oil emulsion cream; an oil-in-water emulsion cream; and a waxy stick (resembling deodorant and perfume sticks). Each of these compositions or formulations includes a carrier for keeping the desired fragrance in the solution. The formulations do not necessarily require complicated equipment and manufacturing steps; and, indeed, they can be prepared with an easy-to-obtain, inexpensive hot plate, a candy thermometer, and a stirrer.

It is an objective of the present invention to provide an odor masking substance that can be used by hunters, fishermen, law enforcement officers and the general public to mask and eliminate noisome odors.

It is another objective of the present invention to provide an odor masking substance that is easy to manufacture and package.

It is yet another objective of the present invention to provide an odor masking substance that masks the noisome odor experienced by hunters during the field dressing of game.

Still yet another objective of the present invention is to provide an odor masking substance for masking noisome odors and which can exude various scents such as wintergreen, spearmint, evergreen, and fruit.

These and other objects, features and advantages will become apparent to those skilled in the art upon a perusal of the following detailed description when read in conjunction with the accompanying drawings.

### BRIEF DESCRIPTION OF THE DRAWINGS

FIG. 1 is a perspective view of the odor masking substance of the present invention illustrating the flexible and portable container for holding the odor masking formulation and from which the odor masking formulation is dispensed.

### DETAILED DESCRIPTION OF THE PREFERRED EMBODIMENT

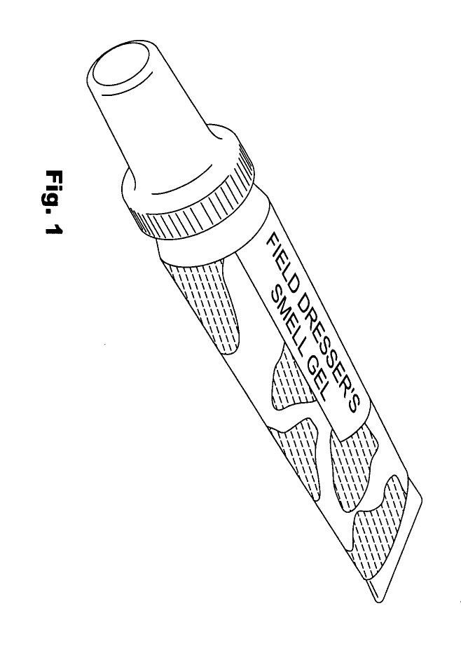

The above objectives can be accomplished by using any of the above-described formulations or compositions. All of the above-described formulations can include various types of fragrances such as pine fragrance (alpha pinene or terpinolene), cedar fragrance (Cedrafix), or cinnamon fragrance (methyl cinnamic aldehyde alpha). In addition, a relatively low cost fragrance such as a lemon-lime fragrance (d-limonene) can be used as well as a mint oil fragrance. Another low cost and effective fragrance that can be use is vanilla. A formulation employing water as the sole carrier is also possible, but the fast evaporation of water will probably make it an ineffective carrier. Thus, a carrier such as propylene glycol or glycerine can be used to maintain the fragrance in solution. As shown in FIG. 1, any of the hereinafter described odor masking formulations can be contained within and dispensed from containers, such as a portable, flexible squeeze tube container of FIG. 1. A small amount of any of the formulations (except for the waxy stick) would be squeezed onto the hunter's fingertip and the topically applied about the hunter's nose for masking the foul odors that are exuded during field dressing game animals.

The odor masking formulation that employs an oil base (petrolatum), commercially known as petrolatum, is widely available from numerous sources.

The second formulation is a glycerine/water gel formulation that includes water 81 parts by weight, glycerine 15 parts by weight, CMC-HV 3 parts by weight, Seakem GP 317 1.0 parts by weight, a fragrance, e.g. vanilla 1-5, and a preservative comprising 0.1-0.5 by weight. CMC-HV is a carboxy methyl cellulose thickener, Seakem GP 317 is a carrageenan gum, and the preservative can be a methyl paraben or a PURAC lactic acid.

A third formulation is a water-in-oil emulsion cream or lotion that comprises ingredients or substances that can be grouped into a Part A and a Part B. The Part A substances include NIMCO 1795 lanolin alcohol 2 parts by weight, mineral oil with a viscosity of 70 cp. and 53 cp. parts by weight, and Emerson 132 stearic acid 3 parts by weight. Part B includes the substances Triethanolamine 1.5 parts by weight, demineralized water 40.3 parts by weight, and methyl paraben 0.2 parts by weight. The preservative methyl paraben is also known as Solbrol M. The method for making this formulation is by the following steps: first heat Part A to 62 degrees centigrade (144 degrees F) and Part B to 65 degrees centigrade (149 degrees F). Then add Part B to Part A and mix while cooling to room temperature. After the emulsion has cooled, a fragrance such as lemon, pine, cedar or cinnamon can be added. If a thicker emulsion is desired, a predetermined amount of CMC-HV can be added to the water of Part B.

A fourth formulation is an oil-in-water emulsion or cream that comprises ingredients or substances that can be grouped in a Part A and a Part B. Part A includes Tegin A 7 parts by weight, paraffin oil, 34 cp 3 parts by weight, Isopropyl myristate 4 parts by weight, and propyl paraben 0.05 parts by weight. Part B includes deionized or distilled water 75.5 parts by weight, methyl paraben 0.15 parts by weight, Germall 115 0.3 parts by weight, Allantoin 0.1 parts by weight, d-Panthenol 0.1 parts by weight, and a thickener Carbopol 934, 0.1 parts by weight. The method or steps for creating the oil-in-water emulsion formulation include, first, heating Part A to dissolve the ingredients or substances, then adding Part A to Part B while stirring. After cooling the desired fragrance can be added.

A fifth formulation is an odor masking item in the form of a waxy stick, such as a deodorant or perfume stick, that includes the following ingredients or substances: PG-3 Myristyl ether 73 parts by weight, propylene glycol 10 parts by weight, water 3 parts by weight, sodium stearate C-1 8 parts by weight, and the desired fragrance 6 parts by weight. The Myristyl ether is a Witconol APM product. The method or steps comprising the formulation of the stick application include heating the propylene glycol and the water to 80-90 degrees centigrade (176 to 194 degrees F), and dissolving the sodium stearate in this solution, stirring until clear. Then the desired fragrance can be added while cooling and continuing the stirring, but before the solidification of the formulation.

Although several preferred embodiments of the invention have been described herein in detail, it is understood that numerous modifications, variations, and alterations may be made thereto by those skilled in the art without departing from the spirit of the invention or the scope of the appended claims.

I claim:

1. An odor masking formulation for masking noisome odors, comprising:
a portable container for storing therein the odor masking formulation and from which the odor masking formulation is dispensed;
the odor masking formulation consisting essentially of:
an oil-based substance; and
a fragrance dispersed throughout the oil-based substance.

2. The odor masking formulation of claim 1 wherein the fragrance is vanilla.

3. The odor masking formulation of claim 2 wherein the fragrance is pine.

4. The odor masking formulation of claim 3 wherein the fragrance is cedar.

5. The odor masking formulation of claim 4 wherein the fragrance is cinnamon.

6. The odor masking formulation of claim 5 wherein the fragrance is lemon-lime.

7. The odor masking formulation of claim 6 wherein the oil-based substance is petrolatum.

8. A glycerine/water gel formulation for masking noisome odors, comprising:
a portable container for storing therein the glycerine/water gel formulation and from which the formulation is dispensed; and
the formulation consisting essentially of water, glycerine, a water-soluble thickener, a preservative and a fragrance.

9. A glycerine/water gel formulation for masking noisome odors, comprising:
a portable container for storing therein the glycerine/water gel formulation and from which the formulation is dispensed;
the formulation consisting essentially of:
81 weight percent water;
15 weight percent glycerine;
3 weight percent CMC-HV;
1.0 weight percent Seakem GP 317;
1 to 5 weight percent fragrance; and
0.1 to 0.5 weight percent preservative.

10. The glycerine/water gel formulation of claim 9 wherein the preservative is methyl paraben.

11. The glycerine/water gel formulation of claim 10 wherein the preservative is PURAC lactic acid.

12. A water-in-oil emulsion cream for masking noisome odors, comprising:
a portable container for storing therein the water-in-oil emulsion cream and from which the formulation can be dispensed; and
the formulation for the water-in-oil emulsion cream consisting essentially of water, a surfactant, hydrocarbon oil, a thickener, a preservative, and a fragrance.

13. A water-in-oil emulsion cream for masking noisome odors, comprising:
a portable container for storing therein the water-in-oil emulsion cream and from which the formulation can be dispensed;
the formulation for the water-in-oil emulsion cream consisting essentially of:
2 weight percent NIMCO 1795 lanolin alcohol;
53 weight percent mineral oil having a viscosity of 70 cp.;
3 weight percent Emerson 132 stearic acid;
1.5 weight percent triethanolamine;
40.3 weight per cent demineralized water; and
0.2 weight percent preservative.

14. The water-in-oil emulsion cream of claim 13 wherein the preservative is methyl paraben.

15. The water-in-oil emulsion cream of claim 14 wherein the method for making the formulation comprises the steps of:
heating the NIMCO 1795 lanolin alcohol, the mineral oil and the Emerson 132 stearic acid to 62 degrees centigrade;
heating the triethanolamine, the demineralized water and the methyl paraben to 65 degrees centigrade;
mixing the triethanolamine, the demineralized water and the methyl paraben to the NIMCO 1795 lanolin alcohol, the mineral oil, and the Emerson 132 stearic acid and cooling to room temperature; and
adding a fragrance after the emulsion cream has cooled.

16. An oil-in-water emulsion cream for masking noisome odors, comprising:
water;
a hydrocarbon oil;
a surfactant;
a thickener;
a preservative; and
a fragrance.

17. An oil-in-water emulsion cream for masking noisome odors, comprising:
7 weight percent Tegin A;
3 weight percent paraffin oil, 34 cp;
4 weight percent isopropyl myristate;
0.05 weight percent propyl paraben;
75.5 weight percent deionized water;
0.15 weight percent methyl paraben;
0.3 weight percent Germall 115;
0.1 weight percent Allantoin;
0.1 weight percent d-Panthenol; and
0.1 weight percent Carbopol 934.

18. The oil-in-water emulsion cream of claim 17 wherein the method for making the formulation comprises the steps of:
heating the Tegin A, the paraffin oil 34 cp, the isopropyl myristate, and the propyl paraben so that they can be dissolved;
adding the Tegin A, the paraffin oil 34 cp, the isopropyl myristate, and the propyl paraben to the deionized water, the methyl paraben, the Germall 115, the Allantoin, Carbopol 934 and the d-Panthenol while stirring; and
adding a fragrance after the emulsion cream has cooled.

19. A waxy stick for masking noisome odors, comprising:
a soft wax;
glycol solvent;
a waxy ester; and
a fragrance.

20. A waxy stick for masking noisome odors, comprising:
73 weight percent PG-3 Myristyl ether;
10 weight percent propylene glycol;
3 weight percent water;
8 weight percent sodium stearate C-1; and
6 weight percent fragrance;

21. The waxy stick of claim 20 wherein the method for making the waxing stick comprises the steps of:
heating the propylene glycol and the water to between 80 degrees and 90 degrees centigrade;
dissolving the sodium stearate C-1 in the propylene glycol and the water;
stirring the propylene glycol, the water and the sodium stearate C-1 until clear; and
adding the fragrance whiling cooling and stirring and before solidification.
